(19) United States
(12) Patent Application Publication
(10) Pub. No.: US 2005/0267550 A1
(43) Pub. Date: Dec. 1, 2005

(54) System and method for medical communication device and communication protocol for same

(75) Inventors: Phillip Hess, Los Angeles, CA (US); Colin Chong, Glendale, CA (US); Ronnie Paul Mounce, Burbank, CA (US); David Y. Choy, San Gabriel, CA (US)

Correspondence Address: FOLEY & LARDNER, 2029 CENTURY PARK EAST, SUITE 3500, LOS ANGELES, CA, 90067, US

(73) Assignee: Medtronic MiniMed, Inc.

(21) Appl. No.: 10/857,579

(22) Filed: May 28, 2004

(51) Int. Cl. A61N 1/08

(52) U.S. Cl. 607/60; 607/32

(57) ABSTRACT

A medical communication device and communication protocols. The medical device may include communication circuitry and a plurality of antennas connected to the communication circuitry. The plurality of antennas may be disposed in the medical device such that the medical device communicates in a plurality of directions. The medical device may communicate with an implantable device implanted with a patient. A protocol for communicating with the implantable device may include monitoring a first antenna in the medical device during a first time slot for a transmitted signal; monitoring a second antenna in the medical device during a second time slot for the transmitted signal after monitoring for the transmitted signal on the first antenna; and generating an acknowledgement when a signal is detected on the first antenna, second antenna or third antenna. The device may be powered by a secondary power supply during communication.

### BACKGROUND

1. Field of the Invention

The present invention relates to communication devices and protocols and, in particular, to communication devices and protocols for implantable medical devices.

2. Description of Related Art

The availability of implantable medical devices has greatly improved mobility for patients who suffer from a variety of ailments and who, in the past, have been forced to remain in close proximity to medical facilities or who have even been forced to remain physically connected to some type of medical apparatus. For example, implantable cardiac pacemakers have revolutionized the treatment of heart disease. Recently, work has begun on implantable insulin pumps to treat diabetes.

Implantable medical devices often operate with a corresponding external device with which the implantable device communicates. The external device may be used to command the implantable device and may be used to send/receive vital information to/from the implantable device. However, many of these implantable devices require that a patient be in direct proximity to an external device, even requiring so much that the patient maintain a specified angle with respect to the external device in order to maintain effective communication. Thus, while implantable medical devices have improved mobility for patients suffering from a wide variety of ailments, there remain situations in which such patients are still bound to the confines of communication devices that are essential for proper operation of their implantable devices.

### SUMMARY

According to embodiments of the present invention, a medical device may include communication circuitry and a plurality of antennas connected to the communication circuitry. The plurality of antennas may be disposed in the medical device such that the medical device communicates in a plurality of directions. The medical device may be configured to communicate with an implantable device, which may be implanted into a patient. In addition, the medical device may communicate with the implantable device via magnetic coupling.

According to embodiments of the present invention, the medical device may communicate with the implantable device using a wavelength in the kilometer range or using a frequency in the kilohertz range. The frequency may be 131 kilohertz. The plurality of antennas may include two antennas or three antennas. The three antennas may be disposed in the medical device such that the medical device communicates in three dimensions. The plurality of antennas may be configured as a plurality of inductors.

According to an embodiment of the present invention, the medical device may further include amplifiers connected to the inductors for driving the inductors. The amplifiers may include enabling and disabling circuitry. The enabling and disabling circuitry may include tri-state circuitry. The amplifiers may be configured to drive only one inductor of the plurality of inductors at a time. The amplifiers may be linear amplifiers or logic buffers. According to an embodiment of the present invention, the medical device may further include a plurality of capacitors for tuning the plurality of inductors.

According to an embodiment of the present invention, a method of communication in a medical device may include providing communication circuitry in the medical device; disposing a plurality of antennas in the medical device; connecting the plurality of antennas to the communication circuitry; and communicating using the medical device. The plurality of antennas may be disposed in the medical device such that the medical device communicates in a plurality of directions. In addition, communicating using the medical device may include communicating with an implantable device.

According to an embodiment of the present invention, a method of communication for a medical device may include monitoring a first antenna in the medical device during a first time slot for a transmitted signal; monitoring a second antenna in the medical device during a second time slot for the transmitted signal after monitoring for the transmitted signal on the first antenna; and generating an acknowledgement when a signal is detected on the first antenna or the second antenna. The method may further include monitoring a third antenna in the medical device during a third time slot for the transmitted signal after monitoring for the transmitted signal on the first antenna and the second antenna; and generating an acknowledgement when a signal is detected on the first antenna, the second antenna or the third antenna. The method may further include designating an antenna on which a signal has been detected as a default antenna for subsequent monitoring and may also include receiving a signal indicating that the acknowledgment was received.

According to an embodiment of the present invention, the first time slot, the second time slot and the third time slot may occur at two-second intervals. Also, the first antenna, the second antenna and the third antenna may be disposed in the medical device substantially perpendicular to one another.

According to another embodiment of the present invention, a method of communication for a medical device may include transmitting a first signal; monitoring a first antenna in the medical device during a first time slot for an acknowledgement that the first signal was received; and monitoring a second antenna in the medical device during a second time slot for an acknowledgement that the first signal was received. The method may further include monitoring a third antenna in the medical device during a third time slot for an acknowledgement that the first signal was received and may also further include receiving a signal indicating that the acknowledgment was received.

According to yet another embodiment of the present invention, a method of communication for a medical device may include monitoring a first antenna in the medical device during a first time slot for a transmitted signal; transmitting a request signal; monitoring a second antenna in the medical device during a second time slot for the transmitted signal after monitoring for the transmitted signal on the first antenna; and generating an acknowledgement when a signal is detected on the first antenna or the second antenna. The method may further include monitoring a third antenna in the medical device during a third time slot for the transmitted signal after monitoring for the transmitted signal on the first antenna and the second antenna; and generating an acknowledgement when a signal is detected on the first antenna, the second antenna or the third antenna.

According to an embodiment of the present invention, a method for supplying power to a circuit may include providing a first power supply for supplying primary power to the circuit; providing a second power supply for supplying secondary power to the circuit; temporarily disabling the first power supply; and temporarily powering the circuit with the second power supply. An amount of noise introduced into the circuit by the second power supply may be less than an amount of noise introduced into the circuit by the first power supply. Also, the first power supply may be a switching regulator. The second power supply may be a battery or a capacitor. The capacitor may be a double layer capacitor. The first power supply may charge the second power supply. Also, temporarily powering the circuit with the second power supply may include temporarily powering the circuit with the second power supply when the circuit is receiving a transmission.

According to an embodiment of the present invention, a system for supplying power to a circuit may include a first power supply for supplying primary power to the circuit; and a second power supply for supplying secondary power to the circuit, wherein the second power supply temporarily supplies power to the circuit when the first power supply is temporarily disabled.

According to an embodiment of the present invention, a method of affixing a transducer to a device may include providing at least one pin fixedly attached to the device; disposing the transducer on the device adjacent the at least one pin; and melting the at least one pin, wherein the at least one pin may be melted such that the at least one pin clasps a portion of the transducer. Providing at least one pin may include providing a plastic pin or a metal pin. Also, melting the at least one pin may includes melting with heat, ultrasonics, friction or vibrations. The method may further include forming a chamber when the transducer is disposed on the device. The chamber may be an acoustic chamber.

### BRIEF DESCRIPTION OF THE DRAWINGS

A detailed description of embodiments of the invention will be made with reference to the accompanying drawings, wherein like numerals designate corresponding parts in the several figures.

FIG. 1 shows a system diagram of an embodiment of the present invention and an environment in which embodiments of the present invention may be used according to an embodiment of the present invention.

FIG. 2 is an external front view of medical communication device according to an embodiment of the present invention.

FIG. 3 shows a schematic diagram of an antenna configuration located within an external device according to an embodiment of the present invention.

FIG. 4 shows a schematic diagram of an antenna configuration located within an external device according to another embodiment of the present invention.

FIG. 5a shows a schematic diagram of a circuit that may be used to drive an antenna according to an embodiment of the present invention.

FIG. 5b shows a schematic diagram of another circuit that may be used to drive an antenna according to an embodiment of the present invention.

FIG. 6a shows a schematic diagram of another circuit that may be used to drive an antenna according to an embodiment of the present invention.

FIG. 6b shows a schematic diagram of another circuit that may be used to drive an antenna according to an embodiment of the present invention.

FIG. 7 shows a schematic diagram of another circuit that may be used to drive an antenna according to an embodiment of the present invention.

FIG. 8 shows a schematic diagram of another circuit that may be used to drive an antenna according to an embodiment of the present invention.

FIG. 9 shows a flow diagram of a communication protocol according to an embodiment of the present invention.

FIG. 10 shows a flow diagram of a communication protocol according to an embodiment of the present invention.

FIG. 11 shows a flow diagram of a communication protocol according to an embodiment of the present invention.

FIG. 12 shows a block diagram of a secondary power supply according to an embodiment of the present invention.

FIG. 13 shows a block diagram of a rear portion of an external device upon which is mounted a transducer according to an embodiment of the present invention.

FIG. 14 shows a side view of a transducer and pins according to an embodiment of the present invention.

FIG. 15 shows a side view of a transducer and melted pins according to an embodiment of the present invention.

### DETAILED DESCRIPTION

In the following description of preferred embodiments, reference is made to the accompanying drawings which form a part hereof, and in which are shown by way of illustration specific embodiments in which the invention may be practiced. It is to be understood that other embodiments may be utilized and structural changes may be made without departing from the scope of the preferred embodiments of the present invention.

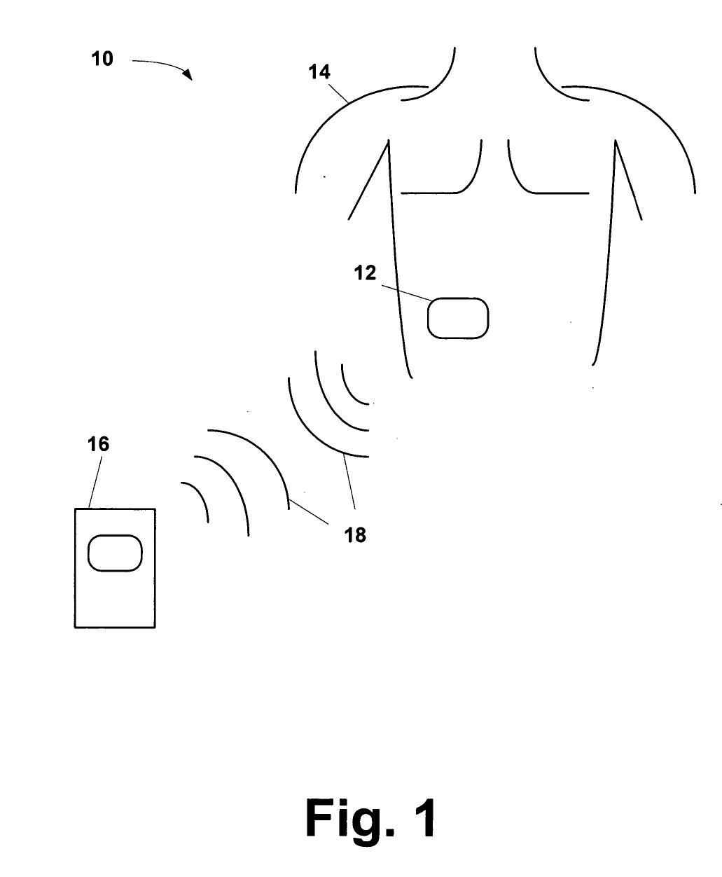

FIG. 1 shows a system diagram of an embodiment of the present invention and an environment in which embodiments of the present invention may be used. The system 10 shown in FIG. 1 includes, but is not limited to, a patient device 12, a patient 14 and an external device 16. The patient device 12 may be an internal, implantable device or an external, non-implantable device. If the patient device 12 is an implantable device, it may be implanted internally within a patient and may be any of a variety of implantable units such as, for example, an implantable insulin pump, a pacemaker or any type of medical device that may be implanted into a patient. In addition, the patient device 12 may include a variety of components to facilitate its operation. For example, if the patient device 12 is an implantable insulin pump, the patient device 12 may also include sensors such as glucose sensors or oxygen sensors, for example.

The external device 16 may communicate with the patient device 12. The external device 16 may be wired to the patient device 12 or may communicate with the patient device 12 via electromagnetic wave propagation or inductive coupling. In the embodiment of the invention shown in FIG. 1, the external device 16 communicates with the patient device 12 via a quasi-static magnetic near field 18, i.e., inductive coupling. Accordingly, the patient 14 is not restricted in mobility. According to an embodiment of the present invention, the patient 14 and, consequently, the patient device 12 may move up to six feet away from the external device 16 and still remain in communication with the external device 16. Other embodiments may permit even greater distance between the patient device 12 and the external device 16. In addition, the external device 16 and the patient device 12 may be configured such that the patient 14 and thus, the patient device 12, may be disposed in any orientation relative to the external device 16 and still maintain effective communication, as will be explained in greater detail below.

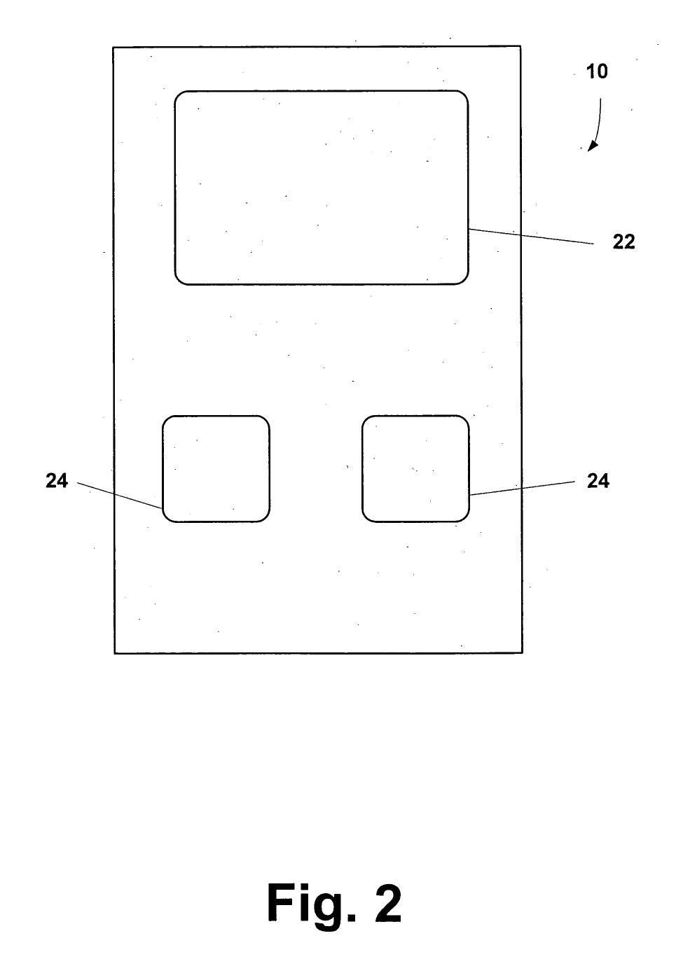

FIG. 2 is an external front view of an external device 20 used to communicate with an implantable device. The external device 20 may be configured in a variety of ways. In the embodiment of the invention shown in FIG. 2, the external device 20 includes a display 22 and keys 24. The display 22 may be implemented in a variety of ways. For example, the display 22 may be an LCD display, and LED display, a vacuum fluorescent display and the like. The keys 24 may be used to enter data and to respond to queries presented in the display 22. In the embodiment of the invention shown in FIG. 2, there are two keys 24. However, the external device 20 may include any number of keys and could be configured with two, three, four or more keys.

The external device 20 may be used in a variety of ways. For example, the external device 20 may be used to communicate with an implantable device or another external device. According to an embodiment of the present invention, the external device 20 may include communication circuitry and other hardware, firmware and software configured to process data transmitted to or received from an implantable device.

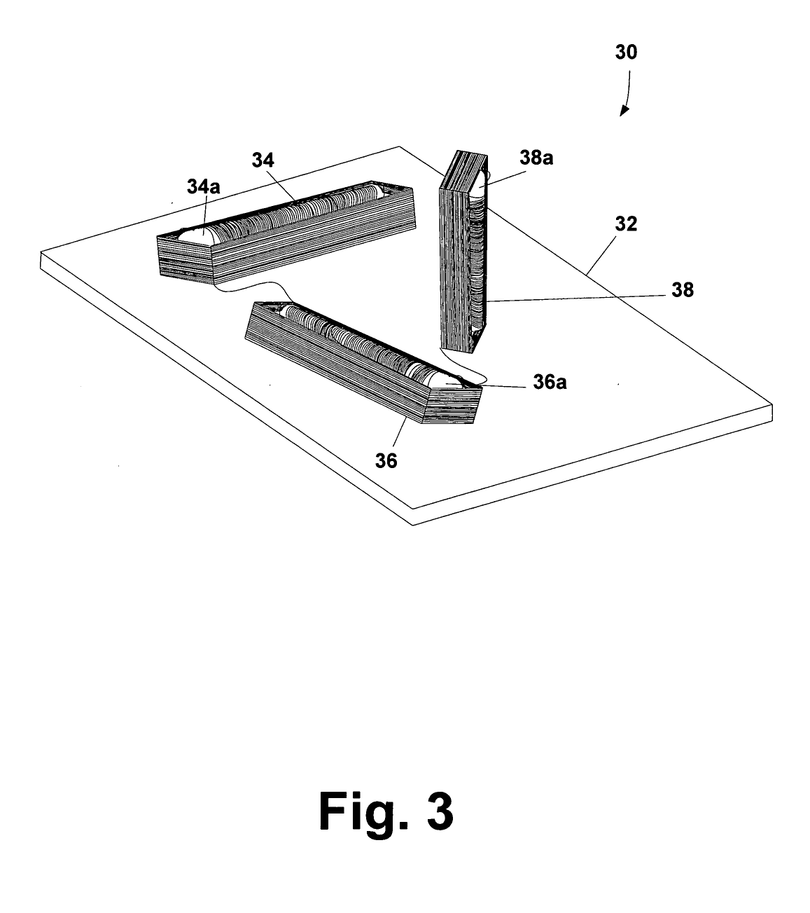

Because the external devices used in embodiments of the present invention, such as the external device 20 shown in FIG. 2 and the external device 16 shown in FIG. 1, for example, may transmit information to and receive information from an implantable device, the external device may be configured with one or more antennas. FIG. 3 shows a schematic diagram of an antenna configuration 30 located within an external device according to an embodiment of the present invention. The configuration 30 shown in FIG. 3 includes, without limitation, a first antenna 34, a second antenna 36 and a third antenna 38. The antennas 34, 36 and 38 may be disposed on a circuit board 32 or may be otherwise disposed within the interior of the external device. The antennas 34, 36 and 38 may communicate with an implantable device.

Any number of antennas may be used with an external device according to embodiments of the present invention. In the embodiment of the invention shown in FIG. 3, the first antenna 34, the second antenna 36 and the third antenna 38 are inductive loop antennas but could be any antennas suitable for transmission and reception. The antennas 34, 36 and 38 may include at least one turn of wire or other traces wound around a geometrical form functioning as a core. In the embodiment of the invention shown in FIG. 3, the cores 34a, 36a, and 38a are cylindrical, but may be, for example, oval, rectangular, square, hexagonal, or any other desired geometry or may even be flat. For example, according to an embodiment of the present invention, if the geometrical cross-section of the antenna is round, the core may be either cylindrical or non-dimensional; the length of the core could effectively be zero since an inductive loop antenna needs only a loop to produce a magnetic field. In addition, the cores 34a, 36a and 38a may be made from air, ferrite or any other suitable core material. The antennas used in the external device may all be wound around one core or, as shown in FIG. 3, may each have their own core.

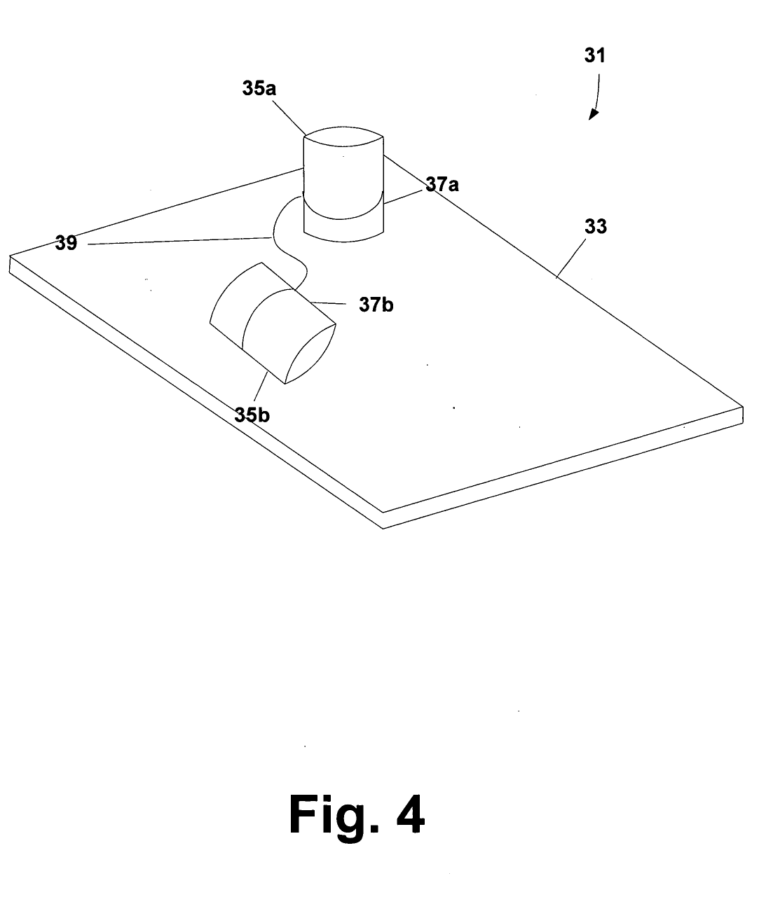

In addition, according to embodiments of the present invention, antennas used in the external device may include several loop antennas, such as inductive loops, for example, with or without cores and connected in series, in parallel or operated independently. For example, FIG. 4 shows a schematic diagram of an antenna configuration 31 located within an external device according to an embodiment of the present invention. The antenna configuration 31 shown in FIG. 4 includes two multiple core antennas with two windings connected in series to produce three antennas. According to the embodiment of the invention shown in FIG. 4, the antennas are cylindrical cores with single windings. The antenna configuration 31 shown in FIG. 4 includes, without limitation, a first antenna that includes, without limitation, a first core 35a and a first winding 37a, and a second antenna that includes, without limitation, a second core 35b and a second winding 37b. The first and second antennas may be disposed on a circuit board 33 or may be otherwise disposed within the interior of the external device. In addition, according to an embodiment of the present invention, the first and second antennas may be include a series connection 39. Examples of antennas that may be used in embodiments of the present invention are discussed in U.S. patent application Ser. No. 10/692,541, entitled “System and Method for Multiple Antennas Having a Single Core,” the contents of which are incorporated by reference herein.

The antennas may be configured to communicate at a variety of frequencies. For example, according to an embodiment of the present invention, the antennas may be configured to communicate at 131 kHz. At frequencies in this range, the antennas may communicate under a variety of conditions. For example, according to an embodiment of the present invention, if an external device is attempting to communicate with an internal device implanted in a patient, at frequencies of about 131 kHz the patient may be up to about six feet away from the external device and still maintain communication.

By way of background, and not of limitation, references to frequency ranges used herein generally follow the following conventions made in the Radio Regulations of the ITU, Article 2, 208, Geneva 19 82, where:

| | | |
|---|---|---|
| 3 to 30 kHz | VLF | Very Low Frequency |
| 30 kHz to 300 kHz | LF | Low Frequency |
| 300 kHz to 3 MHz | MF | Medium Frequency |
| 3 MHz to 30 MHz | HF | High Frequency |

Also, as is known to those skilled in the art, the wavelength, λ, in meters of an electromagnetic wave is given by λ=c/f, where c=3×10⁸ meters/sec and f=frequency of the wave. Thus, an electromagnetic (EM) wave in the VLF to MF frequencies will have a wavelength anywhere from 100 km to 100 meters. As is known in the art, to make an efficient transmitting antenna that radiates EM waves (i.e., creates electromagnetic waves that propagate and travel large distances), the electrical length of the antenna should be on the order of a quarter wavelength, λ/4 or larger, as is common in the art. At frequencies near 100 kHz, the wavelength is 3 km and a quarter wavelength is on the order of 0.75 km, making it generally impractical to build antennas that produce propagating EM waves at this frequency. For a “large loop” antenna, which propagates EM waves and is shaped as a loop of any geometry, such as round, square, hexagonal and the like, one would need a loop perimeter on the order of 0.5λ, equally impractical for small handheld devices.

According to an embodiment of the present invention, a “small antenna” may be chosen to implement a communication link. A “small antenna” may be defined as one that is electrically small compared to a wavelength. A “small loop antenna” is a type of “small antenna.” A “small loop antenna” may be characterized by a loop of turns of wire whose circumference is small compared to a wavelength. The loop may be any of any cross-sectional geometry, such as, for example, round, square, oval and the like, and may or may not have a certain length to it, giving it the shape of a cylinder or square cylinder or hexagonal cylinder, and the like. According to an embodiment of the present invention, the total wire length in the turns may be less than a wavelength to qualify as a “small loop”, although, according to an embodiment of the present invention, the total wire length may be on the order of a wavelength. In an embodiment of the present invention where the total wire length may be on the order of a wavelength, some amount of propagation may occur, making such antennas less “small loop” and more “large loop”. At MF or LF or VLF frequencies, however, winding kilometers worth of turns of wire may be impractical. Thus, a small loop may be defined as one that has a small circumference and a small overall wire length relative to a wavelength. The small loop antenna behaves as a magnetic dipole, generating a “quasi-static” magnetic field: “quasi” because it may be operating at frequencies of 10 kHz or 100 kHz or 1 MHz or the like, and “static” because the magnetic field generated does not propagate.

Patterns generated by a magnetic dipole or by a small loop antenna are generally directional in nature and may require that a receiving antenna be oriented in a specific manner with respect to a transmitting antenna for maximum reception. Inductive coupling refers to antennas that operate using the small loop principle, i.e., they are small compared to a wavelength and do not produce propagating EM waves, but produce quasi-static magnetic fields.

As is known by those skilled in the art, an inductor may be used to produce a magnetic field, and magnetic fields experience loss proportional to the cube of distance, as opposed to the normal path loss of propagating waves, which experience loss proportional to the square of distance. Atmospheric and man made noise is much higher at VLF/LF/MF frequencies than higher bands. At VLF/LF/MF frequencies, the range obtainable is proportional mainly to antenna size, and to achieve ranges of 3 to 9 feet, for example, antennas may be made sufficiently large and bulky. Low frequency communication may be independent of receiver noise specifications. Because the external noise at VLF/LF/MF frequencies is relatively high, receivers at these frequencies may utilize reduced current and power in order to achieve a low noise figure. Higher frequencies generally have much higher receiver power dissipation than VLF/LF/MF frequency receivers since they may be limited by receiver noise.

Because propagation at lower frequencies may be reduced, minimal, or non-existent, phase cancellations of traveling EM waves may be minimal or non-existent and any interfering signal causing a loss of RF link between two devices can be avoided by simply moving away from the interferer and/or moving the two devices closer together. Thus, using multiple antennas to “cover” a sphere of a given number of feet may facilitate coverage.

Also, the SAR, i.e., the “Specific Absorption Rate,” the extent to which the human body absorbs a radiating or non-radiating electromagnetic field, generally increases with increasing frequency, for various kinds of tissue, such as, muscle, fat and the like. Thus, lower frequencies may experience less absorption, resulting in less loss and less attenuation as they travel through or near the human body.

For an implant or for a medical device mounted near the body, the use of lower frequencies may result in lower receive power dissipation, low attenuation by body tissue, low loss through the body in both directions, eased receiver noise figure requirements, and ease of obtaining license-free operation, since many countries allow LF operation below a certain number of kHz without a license below a certain power level. Multiple antennas used in low frequency RF systems may be used to avoid the inherent, unavoidable nulls obtained from transmitting and receiving a quasi-static magnetic field.

The quasi-static magnetic fields produced and received by antennas such as the inductive loop antennas 34, 36 and 38 shown in the embodiment of the invention shown in FIG. 3 are directional in nature and have nulls and peaks. Accordingly, the use of several antennas disposed with various orientations relative to each other permits a telemetry scheme to cover a given range irrespective of the orientation of a transmit or receive antenna. According to an embodiment of the present invention, three antennas may be oriented in such a way, such as that shown in FIG. 3, for example, to cover nulls in three directions, such as the x, y and z directions of three dimensional space, for example. However, embodiments of the invention are not limited to three antennas and one, two, three or more antennas may be used and oriented with respect to each other in any manner suitable to satisfy parameters specified for a system.

The inductive loop antennas 34, 36 and 38 shown in FIG. 3 may be oriented in a variety of ways. According to the embodiment of the invention shown in FIG. 3, the inductive loop antennas 34, 36 and 38 are mounted perpendicularly in separate planes with respect to each other and, thus, are disposed in such a way as to communicate in three directions. The inductive loop antennas 34, 36 and 38 need not have 90 degrees of separation, however. For example, if more than three antennas are used, the antennas may be oriented at specified angles such that the antennas communicate in directions convenient to a user or such that the antennas cover nulls in as many directions as desired. For example, according to an embodiment of the present invention, six antennas may be used for communication. If six antennas are used, two antennas may be positioned in each orthogonal plane in free space. The antennas in each plane may be oriented 60 degrees apart from each other.

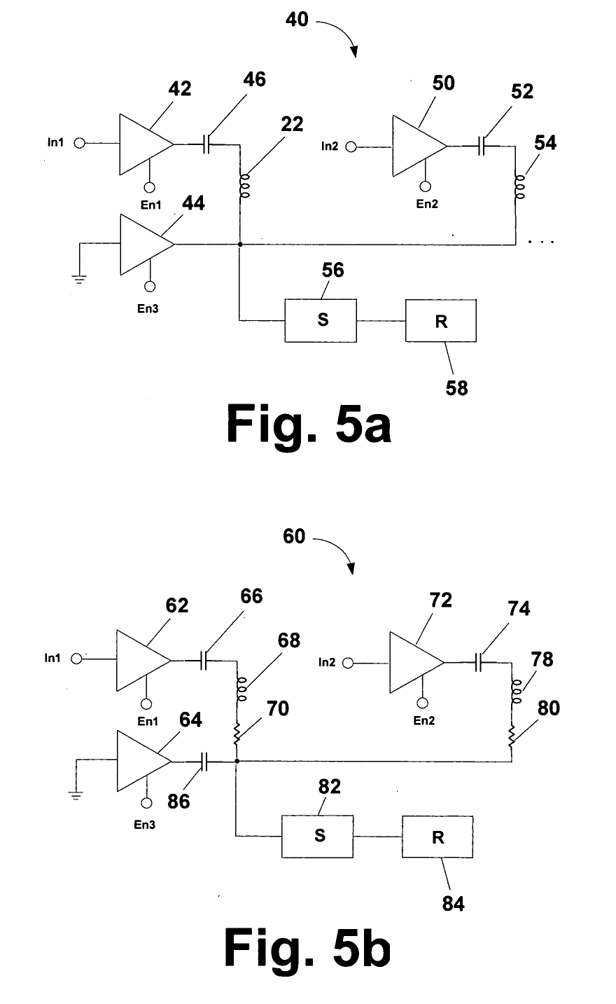

According to an embodiment of the present invention, the antennas may be driven or enabled separately and time-multiplexed to generate or receive a known magnetic field pattern, although more than one antenna may be enabled or driven concurrently. FIG. 5a shows a schematic diagram of a circuit 40 that may be used to drive an antenna according to an embodiment of the present invention. The circuit 40 includes, but is not limited to, a first driver 42, the output of which is connected to a first end of a first capacitor 46. A second end of the first capacitor 46 is connected to a first end of a first inductor 48. A second end of the first inductor 48 is connected to a second end of a second inductor 54. A first end of the second inductor 54 is connected to a second end of a second capacitor 52. A first end of the second capacitor 52 is connected to an output of a second driver 50. The second end of a first inductor 48 and the second inductor 54 may also be connected to an output of a third driver 44. According to the embodiment of the invention shown in FIG. 5a, the circuit 40 is a single-ended mode driving circuit.

The first, second and third drivers 42, 50 and 44, respectively, may be a digital buffer performing a logic function, a power amplifier, a small signal amplifier, another type of linear amplifier or the like. In operation, a signal may be applied to an input of the first driver 42. The signal applied to the input of the first driver 42 may be a digital signal or an analog signal and may be modulated or unmodulated. The signal applied to the input of the first driver 42 may be identical to a signal applied to an input of the second driver 50.

According to the embodiment of the present invention shown in FIG. 5a, the first inductor 48 and the second inductor 54 function as antennas. The first capacitor 46 and the second capacitor 52 may be a single capacitor or may be a plurality of capacitors in series or in parallel. The first capacitor 46 and the second capacitor 52 may be used to series-tune the first inductor 48 and the second inductor 54, respectively, to a desired frequency. The desired frequency may or may not be a carrier frequency. However, according to embodiments of the present invention, the circuit 40 may be used without capacitors or with capacitors that do not tune the inductors to the frequency of RF communication. According to some embodiments of the present invention, the antennas may be so large or the range so short that capacitors may not be needed.

The first, second and third drivers 42, 50 and 44 each may include an enable pin, which, when deactivated, places the output of the first, second and third drivers 42, 50 and 44 into a high impedance state. Thus, for example, when the enable pin of the first driver 42 is deactivated, the first capacitor 46 and the first inductor 48 are effectively disconnected from any input signal existing on the input of the first driver 42. In addition, any current path through the first inductor 48 resulting from an input signal on the first driver 42 is disabled. An input signal may be applied to the input of the second driver 50 and the enable pin of the second driver 50 may be activated while deactivating the enable pin of the first driver 42. Thus, in this configuration, the only current path will be through the second capacitor 52 and the second inductor 54, the combination of which may be tuned to a desired frequency.

Accordingly, by using the enable pins on the respective drivers, a particular antenna may be chosen for transmission or reception. As shown in the embodiment of the invention in FIG. 5a, the input of the third driver 44 is grounded. Because the second sides of the first inductor 48 and the second inductor 54 are connected to the output of the third driver 44, enabling the third driver 44 will close a current path to ground. If the third driver 44 is disabled, its output will be put into a high impedance state and transmission will not be possible.

Another single-ended mode driver circuit 60 according to an embodiment of the present invention is shown in FIG. 5b. The circuit 60 shown in FIG. 5b is similar to the circuit 40 shown in FIG. 5a and includes a first driver 62, a first capacitor 66, a first inductor 68, a second driver 72, a second capacitor 74, a second inductor 78 and a third driver 64. In addition, the circuit 60 shown in FIG. 5b includes a first resistor 70 and a second resistor 80. The first resistor 70 and the second resistor 80 may be placed in series with the first inductor 68 and the second inductor 78, respectively, to lower the Q of the resonant circuit. In addition, a third capacitor 86 may be placed in series with the return path through the third driver 64 to aid in the tuning or matching of the antenna to the receiver.

In both the circuit 40 shown in FIG. 5a and the circuit 60 shown in FIG. 5b, the first and second inductors may function as the antennas themselves or they may function as transformer primaries which induce current in the secondary coils designed to produce a magnetic field. In the embodiment of the invention in which the inductors are configured as transformer primaries, secondary coils acting as antennas may be tuned with a capacitor or may be untuned.

According to an embodiment of the present invention, a single antenna may be designated as the receive antenna, although many antennas may be enabled at the same time. Thus, for example, to receive on only one antenna such as the second inductor 54 in the circuit 40 of FIG. 5a, the first driver 42 would be disabled while the second driver 50 and the third driver 44 would both be enabled. A signal on the input of the second driver 50 may assume a constant DC value, thus causing the output of the second driver 50 to appear as a low impedance node to AC ground and enabling a path for current to flow through the second capacitor 52 and the second inductor 54. The first inductor 48 sees a high impedance node to ground at the output of the first driver 42 because the first driver 42 has been disabled. Thus, the first inductor 48 does not receive a signal comparable to the signal received by the second inductor 54.

In both FIG. 5a and FIG. 5b, the output of a received signal may be fed to a first switch block, such as the first switch block 56 shown in FIG. 5a or the first switch block 82 shown in FIG. 5b, which may be either a switch or a short-circuit or some type of current path to a second block which may function as a receiver such as, for example, a second block 58 shown in FIG. 5a or the second block 84 shown in FIG. 5b. The second block 58 in FIG. 5a and the second block 84 in FIG. 5b may be networks for matching an antenna impedance to receiver amplifiers or may have some matching components present in the antenna circuitry. For example, the third capacitor 86 shown in FIG. 5b may be part of a matching network in the second block 84. By including the third capacitor 86 in the return transmit path, transmit and receive tuning of the first inductor 68 and the second inductor 78 will not differ significantly due to the effects of matching the third capacitor 86. However, according to embodiments of the present invention, the third capacitor 86 could actually be any element that functions a part of a coupled, tuned circuit. Thus, in a transmit mode, any element could be used to facilitate tuning requirements as the circuit switches from a transmit to receive mode. For example, capacitors, inductors and resistors may be used.

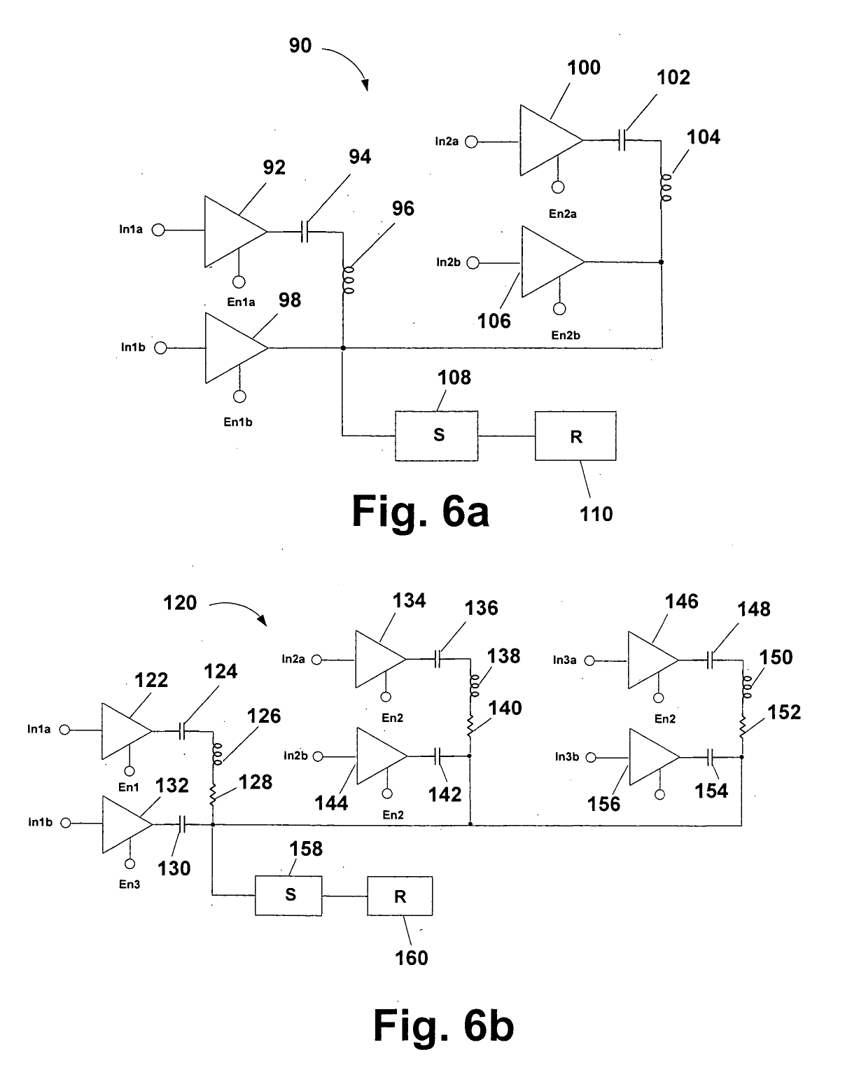

A differential mode driving circuit for driving current through antennas for a transmission according to an embodiment of the present invention is shown in FIG. 6a. The circuit 90 shown in FIG. 6a includes, but is not limited to, a first driver 92, the output of which is connected to a first end of a first capacitor 94. A second end of the first capacitor 94 is connected to a first end of a first inductor 96. A second end of the first inductor 96 is connected to an output of a second circuit element 98 and also to a second end of a second inductor 104. The second end of the second inductor 104 is also connected to an output of a fourth driver 106. A first end of the second inductor 104 is connected to a second end of a second capacitor 102. A first end of the second capacitor 102 is connected to an output of a third driver 100. As was the case with the circuit 40 shown in FIG. 5a and the circuit 60 shown in FIG. 5b, the first, second, third and fourth drivers 94, 98, 100 and 106 shown in FIG. 6a may be digital buffers, linear amplifiers, power amplifiers, other linear amplifiers or the like. Signals on the input of the first, second, third and fourth drivers 92, 98, 100 and 106 may be digital or analog signals at a particular carrier frequency.

In an ideal differential mode operation, a signal on the input of the first driver 92 is 180° out of phase with respect to a signal on the input of the second driver 98. In addition, a signal on the input of the third driver 100 is 180° out of phase with respect to a signal on the input of the fourth driver 106. Accordingly, there would be a 2× gain due to the differential drive across the first inductor 96 and the second inductor 104, as is common in differential amplifiers. The first inductor 96 and the second inductor 104 may be configured as antenna coils themselves or may be the primary coils in a transformer having secondary coils. The secondary coils may be tuned with a capacitor or may be untuned and used as antennas. Although, in ideal conditions, the signals at the input of the first driver 92 and the second driver 98 as well as the signals on the input of the third driver 104 and the fourth driver 106, are 180° out of phase, these signals may not be 180° out of phase and, thus, some differential gain will be lost. According to an embodiment of the present invention, the signal on the input of the first driver 92 may be equivalent to a signal on the input of the third driver 100, although this may not always be the case. For transmission on any given antenna, the enable signal for that antenna may be activated while the enable signals of the other antennas may be deactivated. For simplicity, according to some embodiments of the present invention, the enable pins on the first driver 92 and the second circuit element 98 may be controlled by the same signal. Likewise, the enable pins of the third circuit element 100 and the fourth driver 106 may be controlled by the same signal.

According to an embodiment of the present invention, the differential signal may be a modulated signal. Because transmitted/received signals may be in the VLF/LF/MF range, electronics may be incorporated that will produce an inverted copy of an analog/digital modulated RF signal at these frequencies without distortion, producing a differential pair without consuming excessive power. The frequency range used may be low enough to allow modulation to occur first, then inversion, then application of the two inverted signals to the antenna driver inputs. Thus, the two 180 degree signals may be produced from a single modulated signal.

According to an embodiment of the present invention, during reception, the enable lines may be activated for the desired antenna and a constant DC voltage may be applied to the input of the particular driver for that particular antenna while the enable lines for antennas whose use is not desired are deactivated. Thus, in this configuration, a low impedance, closed path may exist from the driver through its associated capacitor and inductor, in a manner similar to the single-ended case. In addition, a high impedance at the disabled drivers prevents closing of a tank circuit loop for antennas whose use is not desired, also similar to the single-ended case. A receive signal may be extracted at the common node at the second ends of the first inductor 96 and the second inductor 94, respectively, and sent through a first block 108, which may be a switch, a short-circuit or some other current path to a second block 110, which may be a receiver circuit.

According to an embodiment of the present invention, resistors may be placed in series with antennas to lower the effective Q of the circuit. For example, as can be seen in FIG. 6b, a first resistor 128 may be placed in series with a first inductor 126, a second resistor 140 may be placed in series with a second inductor 138, and a third resistor 152 may be placed in series with a third inductor 150. In addition, first, second and third capacitors 130, 142 and 154, respectively, or other elements having particular impedances may be placed in series with a closed path to preserve antenna-tuning accuracy between transmit and receive configurations. The capacitors 130, 142 and 154 may be part of a matching circuit in the receiver 160, or may simply be inserted to change the tuning characteristics of a given antenna. The circuit 120 is configured to drive three antennas.

According to an embodiment of the present invention, all drivers that can be enabled or disabled, such as, for example, the first, second and third drivers 92, 198 shown in FIG. 6A may include tri-state circuitry or may simply be a driver such as an amplifier or buffer followed by a switch. According to an embodiment of the present invention, the drivers may be tri-stateable bus buffers.

According to an embodiment of the present invention, to increase driver power, several of the drivers may be used in parallel with one another, with each input on the driver connected to an input signal and each output of the driver wired together. According to this embodiment of the present invention, a lower effective output impedance is achieved for the drivers and drive current capability is increased.

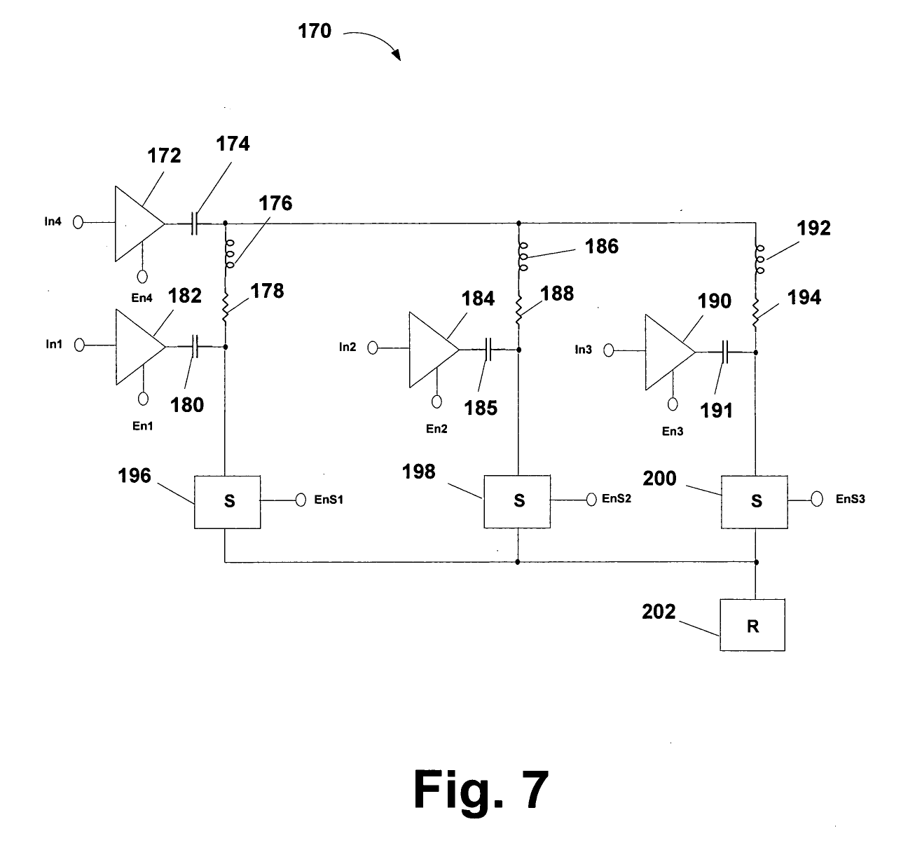

FIG. 7 shows a schematic diagram of a drive circuit 170 according to another embodiment of the present invention. The drive circuit 170 shown in FIG. 7 includes, but is not limited to, a first buffer 172, the output of which is connected to a first end of a first capacitor 174. A second end of a first capacitor 174 is connected to a first end of a first inductor 176. A second end of the first inductor 176 is connected to a first end of a first resistor 178. A second end of the first resistor 178 is connected to a first end of a second capacitor 180. A second end of the second capacitor 180 is connected to an output of a second driver 182. The first end of the second capacitor is also connected to the first end of a first switch 196. The combination of the second driver 182, the second capacitor 180, the first inductor 176, the first resistor 178, and the first switch 196 is connected in parallel to identical circuit combinations as shown in FIG. 7. The second circuit combination includes a third driver 184, a third capacitor 185, a second inductor 186, a second resistor 188 and a second switch 198. The third circuit combination includes a fourth driver 190, a fourth capacitor 191, a third inductor 192, a third resistor 194, and a third switch 200. The outputs of the switches 196, 198 and 200 are all connected together and also connect to a receiver circuit 202.

In the drive circuit 170 show in FIG. 7, each antenna, manifested as the first inductor, the second inductor, and the third inductor 176, 186 and 192, respectively, each have a corresponding receive switch 196, 198 and 200. In addition, each antenna has a corresponding driver in the form of the second, third and fourth drivers 182, 184 and 190. A common driver 172 is used for all antennas. The switches 196, 198 and 200 may be activated by enable lines. Accordingly, the embodiment of the invention shown in FIG. 7 allows differential operation along any given antenna; the first driver 172 may be placed into an enabled mode during transmission, reception and any time during which there is no communication activity. For example, during transmission, using the second inductor 186 as an antenna, the first driver 172 may be enabled and a signal may be applied to the first driver 172, the third driver 184 or both. The signals may or may not be 180 degrees apart. The second driver 182 and the fourth driver 190 may then be disabled as may the second switch 198 so that the transmission current path exists only through the first driver 172 and the third drive 184.

During reception of a signal, the second switch 198 may be enable while a constant DC signal may be applied to both the first driver 172 and the third driver 184. The first driver 172 and the third driver 184 may be enabled, allowing a receive signal to pass to the receiver block 202. The other antennas will see low impedance nodes in both the transmit and receive modes and will not contribute to the circuit while disabled. The switches 196, 198 and 200 may be implemented with simple FET gates, relays, analog switches and the like.

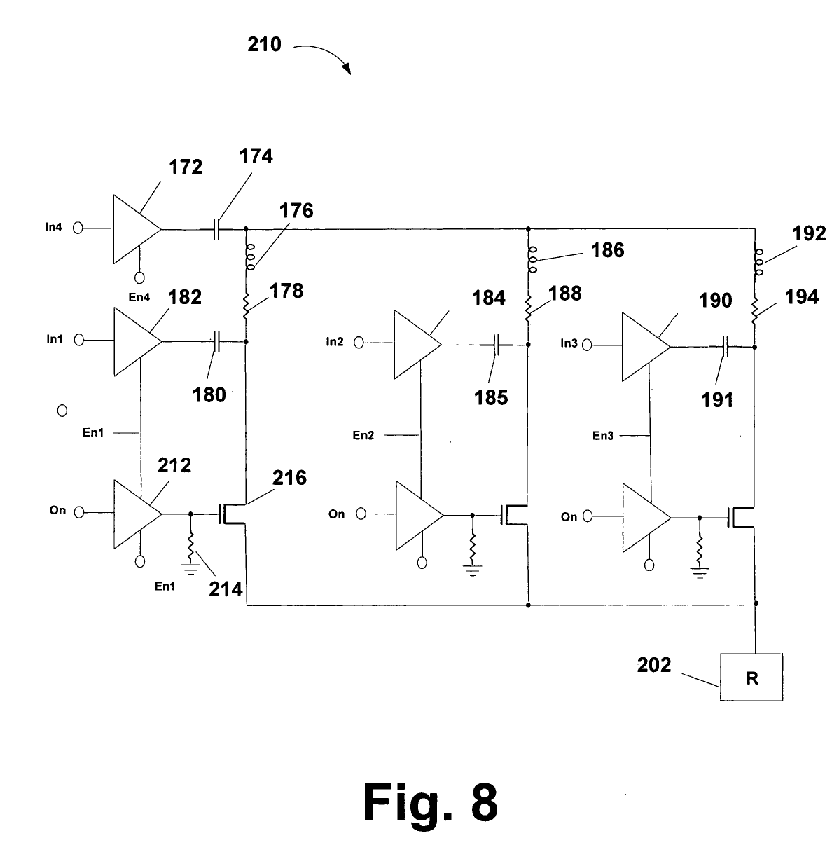

The embodiment of the invention shown in FIG. 8 is essentially identical to the embodiment of the invention shown in FIG. 7 except that the switches 196, 198 and 200 in FIG. 7 have been replaced with drive circuits. For example, the first switch 196 in FIG. 7 has been implemented in FIG. 8 as a first driver 212, a first resistor 214, and a first FET 216. Similar circuits replace the second and third switches 198 and 200. In FIG. 8, to activate the first antenna 176, for example, an “on” signal may be applied to the first driver 212, thereby turning on the FET 216 to allow transmission/reception using the first antenna 176.

Embodiments of the present invention may be configured with hardware, software and firmware that operate to generate signals that enable and disable the drivers driving the inductors. For example, according to an embodiment of the present invention, software or firmware operating the external device may be configured to generate signals that dictate that a particular antenna be enabled or disabled during a particular communication time slot. Accordingly, the signals generated by software or firmware may be sent to a bus. One or more registers may be connected to the bus. Outputs of the register may then be connected to enable lines on the drivers and the signals received by the register from the bus may be applied to the drivers to enable or disable them as necessary according to the software or firmware. Embodiments of the present invention may also use other schemes to enable or disable the drivers.

According to embodiments of the present invention, a variety of communication protocols may be implemented for transmitting and receiving. Referring back to FIG. 1, the external device 16 may be implemented with one, two, three or more antennas when communicating with the patient device 12. Likewise, the patient device 12 may be implemented with one, two, three or more antennas when communicating with the external device 16. By way of example, and not by way of limitation, the following discussion of communication protocols assumes that the external device 16 has been implemented with three antennas oriented in such a way that transmission and reception is possible in three physical dimensions. In addition, by way of example and not by limitation, the following discussion of communication protocols assumes that the patient device 12 has been implemented with one antenna, although the patient device 12 may be implemented with any number of antennas. In addition, although FIG. 1 shows one external device 16 communicating with one patient device 12, any number of external and internal devices may be used for communication. For example, according to an embodiment of the present invention, one internal device may communicate with two, three or more external devices.

According to an embodiment of the present invention, if the patient device 12 is an implanted device having a single antenna and the external device 16 has multiple antennas, power consumption may be reduced in the implanted device. The external device 16 may have power consumption requirements that are less stringent than the patient device 12 if the patient device 12 is implanted because the power source of the external device 16 is more easily replaceable than the power source of an implanted device. According to an embodiment of the present invention, an implanted device may transmit only when necessary since transmission may be expensive in terms of power drained from an implanted power source at very low, low and medium frequencies.

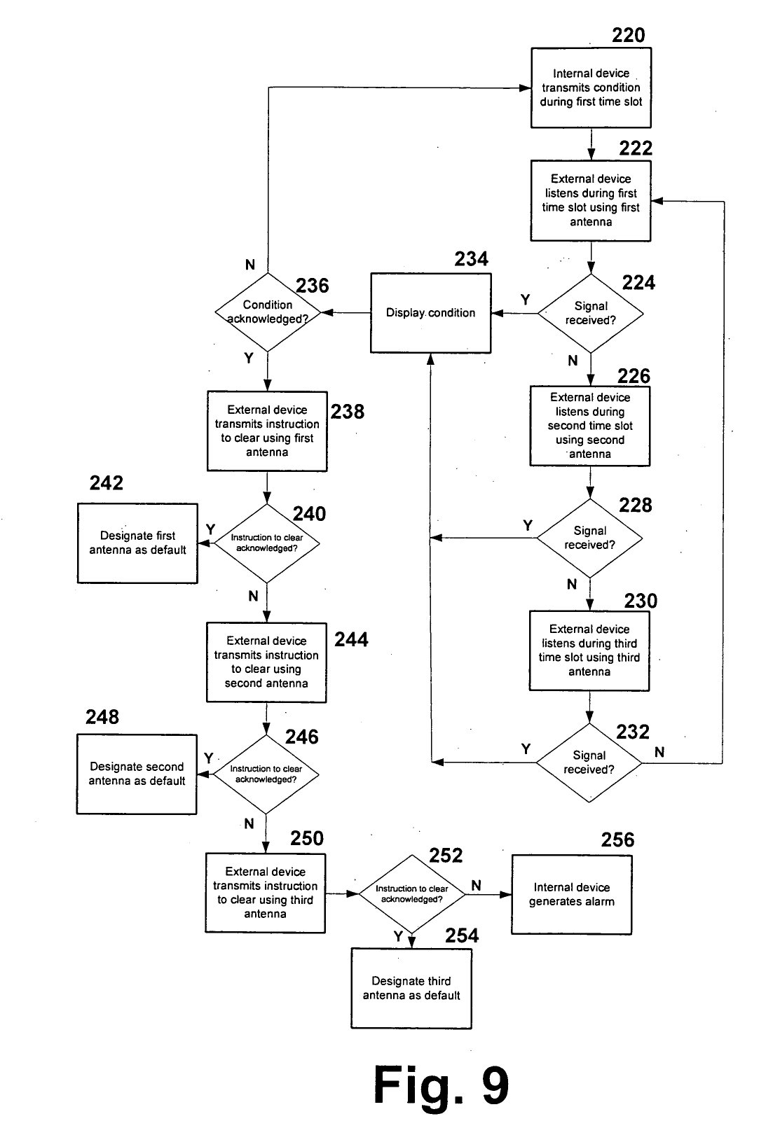

FIG. 9 shows a flow diagram 40 of a communication protocol according to an embodiment of the present invention. The communication protocol shown in FIG. 9 may be used in a variety of circumstances. For example, according to an embodiment of the present invention, when an alarm condition or other condition arises in the internal device, the internal device may generate a series of outbound transmissions on a next available communication timeslot. In addition, the external device may be configured to receive transmission during these designated time slots and may select a different antenna on which to receive transmissions on each successive timeslot cycle. By rotating through each antenna implemented in the external device, the external device is able to take advantage of multiple opportunities to recover the internal device's transmission. Moreover, the external device may identify the antenna that renders the best reception. When a transmission is received and validated, the antenna on which the transmission was received may be designated as a preferred or default antenna. As such, the default antenna may be used as the first antenna selected in any additional attempts to communicate. If the relative positions of the internal device and the external device have not shifted, the default antenna will likely be the best choice for communication. If, however, the positions of the internal device and the external device have shifted, the embodiment of the communication protocol detailed in FIG. 9 advances through each antenna until the antenna best suited for communication is found.

At step 220, the internal device transmits an alarm condition or other condition during a first timeslot. A condition may be an alarm condition, or other type of exception condition. For example, if the internal device includes an implantable glucose sensor, the internal device may transmit a glucose value and/or a glucose-related alarm condition on a particular timeslot. According to an embodiment of the present invention, the timeslot for an outgoing transmission may occur every one-minute in three second intervals. In other words, the internal device may transmit a condition at one-minute, one-second; one-minute, three-second; and one-minute, five-second timeslots. At step 222, the external device listens during the first timeslot using a first antenna for a transmission from the internal device.

At step 224, a determination is made as to whether a transmission from the internal device is received by the external device. If the transmission is received, the external device displays the condition received from the internal device at step 234. If the external device does not receive the signal, then, at step 226, the external device listens during a second timeslot using the second antenna. If the signal is received at step 228, the external device again displays the condition at step 234. If the signal is not received at step 228, the external device then listens during a third timeslot using a third antenna. In a similar manner, at step 232, if a signal is received by the external device, the external device displays the condition received at step 234. If the signal is not received, the first antenna is used again during the next available timeslot for reception of the transmission from the internal device. After a condition is displayed by the external device at step 234, the external device may wait for a user to acknowledge the condition.

If the user does not acknowledge the condition at step 236, the internal device will continue to transmit the condition. If a user does acknowledge the condition at step 236, then the external device will transmit an instruction to clear the condition to the internal device using a first antenna at step 238. If the clearance instruction is acknowledged by the internal device at step 240, the first antenna will be designated as the default antenna for transmission and reception at step 242. If the internal device does not acknowledge the clear instruction at step 240, the external device will transmit a clear instruction using a second antenna at step 244. If the internal device acknowledges this transmission of the clear instruction at step 246, the second antenna will be designated as the default antennae at step 248. If the internal device does not acknowledge the instruction to clear the condition at step 246, at step 250 the external device will transmit an instruction to clear using the third antenna.

If the internal device acknowledges the instruction to clear at step 252, the third antenna will be designated as the default antennae at step 254. If, however, this attempt to clear the condition is not acknowledged by the internal device at step 252, the internal device may generate a separate alarm at step 256. The separate alarm generated by the internal device at step 256 may be any of a variety of alarms, including an audible alarm.

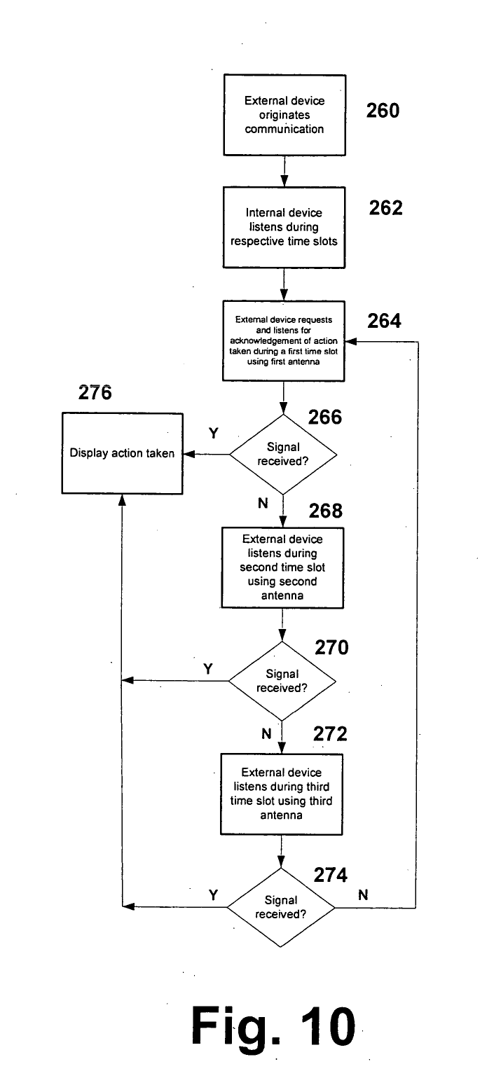

FIG. 10 shows a flow diagram of a communication protocol according to another embodiment of the present invention. The embodiment of the invention shown in FIG. 10 is somewhat similar to the embodiment of the invention shown in FIG. 9. However, in the embodiment of the invention shown in FIG. 10, the external device originates communication at step 260. For example, if the implantable device is an implantable insulin pump, and the patient would like to start a bolus of insulin, the patient may command the external device to send a signal to the internal device to start a bolus. Accordingly, at step 262, the internal device listens to commands from the external device during the respective timeslots. After the internal device has received the transmission from the external device, whatever action is necessary as designated by the external device's communication may be taken by the internal device.

At step 264, the external device may request and listen for an acknowledgement of the action taken from the internal device during a first timeslot using a first antenna. At step 266, a determination is made as to whether an acknowledgement signal from the internal device is received. If an acknowledgement is received by the external device from the internal device, the external device may display that the requested action has been taken at step 276. If the external device has not received an acknowledgement signal from the internal device, then, at step 268, the external device listens for the acknowledgement signal during a second timeslot using a second antenna. At step 270, a determination is made as to whether the signal has been received. If the acknowledgement signal has been received, the external device displays that the requested action has been taken at step 276. If the acknowledgement signal has not been received, the external device listens during a third slot using a third antenna at step 272 for the requested acknowledgement signal.

At step 274, a determination is yet again made as to whether the acknowledgement signal has been received. If the acknowledgement signal has been received, the external device may display that the requested action has been taken at step 276. If the acknowledgement signal has not been received, the external device may again, at step 264, query each successive antenna during each successive timeslot until an acknowledgement signal has been received.

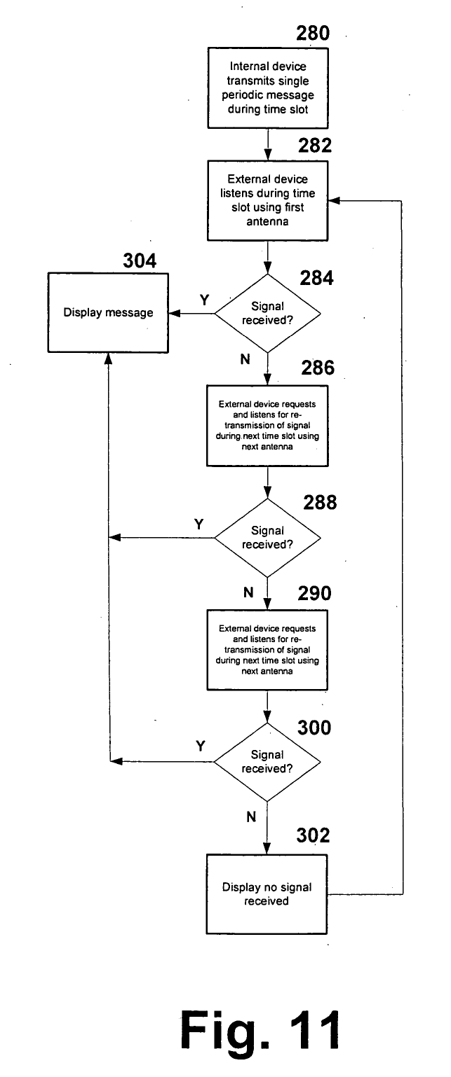

FIG. 11 shows a communication protocol according to yet another embodiment of the present invention. The embodiment of the invention shown in FIG. 11 may combine some of the elements of the embodiment of the invention shown in FIG. 9 and the embodiment of the invention shown in FIG. 10. The communication protocol shown in FIG. 11 may be used, for example, in communications in which a periodic transmission signal originates with an implantable device. According to an embodiment of the present invention, an implantable device may transmit a single signal during a designated, periodic time slot. In addition, an external device may try to capture the signal transmitted by the implantable device by listening, i.e., permitting reception, during the same time slot. If the signal transmitted by the implantable device is not received by the external device, the external device may then solicit the internal device for the transmission. In other words, the external device may request that the internal device transmit the message again, rotating through the plurality of antennas in the external device on each successive attempt to recover the signal transmitted by the internal device. When the transmitted signal is received on a particular antenna, that antenna may be set as a default antenna for transmission and reception.

According to the embodiment of the invention shown in FIG. 11, at step 280, an internal device may transmit a single periodic message during a particular timeslot. At step 282, the corresponding external device may be listening during this timeslot using a first antenna. At step 284, a determination is made as to whether the signal transmitted by the internal device has been received by the external device. If the signal has been received, then the external device may display the message received in the transmission of step 304. If the signal has not been received at step 284, then the external device may request that the internal device retransmit the signal. The external device may listen for the retransmitted signal during the next timeslot using a next antenna.

At step 299, another determination is made as to whether the signal that has retransmitted by the internal device has been received. If the signal has been received, then the external device may display the message received in the transmission in step 304. If the signal has not been received, then the external device may again request that the internal device retransmit the signal while listening for the retransmission of the signal during the next timeslot using a next antenna. At step 300, another determination is made as to whether the retransmitted signal from the internal device has been received by the external device. If the signal has been received, then the external device may display the message received in the transmission in step 304. If the signal has not been received, then at step 302, the external device may display that no signal has been received from the internal device and may jump back to step 282 to initiate another listening and request for transmission cycle.

The external device may step through the cycle of listening for a transmission of a signal from the internal device and requesting that the internal device retransmit the signal when no signal has been received as many times as desired. For example, according to an embodiment of the present invention, the external device may step through the cycle three times in an attempt to receive a signal transmitted by the internal device. If no signal is received after the third iteration of the cycle, the external device may stop requesting that the internal device retransmit the signal. The external device may then display that no signal has been received from the internal device and may notify a user that it will not request retransmission from the internal device until the patient requests that the external device do so. When a patient does request that the external device solicit the internal device for retransmission of a signal, the external device may step through the cycle again a desired number of times as shown in FIG. 11.

Embodiments of the present invention may be powered in a variety of ways. For example, according to an embodiment of the present invention, the external device may be plugged into and receive its power from a standard AC wall outlet. According to another embodiment of the present invention, the external device may be powered by one or more batteries, such as a single AA battery, for example. Moreover, the battery voltage may be up-converted by a switching regulator or switching power supply.

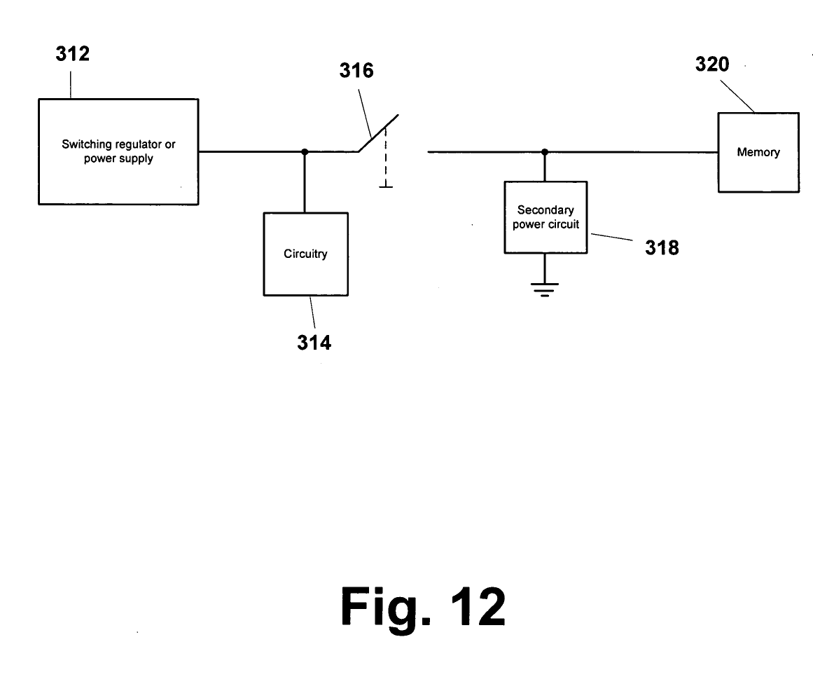

Noise from the switching regulator or switching power supply may have an effect on the transmission and/or reception capabilities of embodiments of the present invention. Accordingly, an embodiment of the present invention shown in FIG. 12 may include a secondary power circuit 318. The secondary power circuit 318 shown in FIG. 12 may be used for a variety of reasons. For example, the secondary power circuit 318 may be used to provide a relatively noise-free power source to power circuitry in the external device during noise-sensitive telemetry periods and to enhance telemetry range. In addition, the secondary power circuit 318 may be used to provide power to keep static memory within the external device powered for an extended period of time.

According to an embodiment of the present invention, the output of the switching regulator or power supply 312 may be connected to circuitry 314 and the first side of a switch 316. A second side of the switch 316 may be connected to the secondary power circuit 318 and memory 320 located within the external device. According to an embodiment of the present invention, the secondary power circuit 318 may be charged by the switching regulator or switching power supply 312 when the switching power supply 312 is available. By maintaining the switch 316 in a normally closed position, the secondary power circuit 318 may be charged by the switching regulator or power supply 312.

According to an embodiment of the present invention, if the switching power supply 312 should become unavailable or, for example, is intentionally shut down, the circuitry 314 and the memory 320 may be supplied with power by the secondary power circuit 318. For example, according to an embodiment of the present invention, during sensitive telemetry periods when the external device is transmitting or receiving, the noise from the switching regulator or power supply 312 may interfere with communication and may degrade performance of the device. Accordingly, during these periods, the switching regulator or switching power supply 312 may be shut-down or disabled. Power to other circuitry 314, which may include, for example, circuits used for transmitting and/or receiving data, and power to memory 320 may then be supplied by the secondary power circuit 318. By shutting down the switching regulator or switching power supply 312 when the external device is in a communication mode and using low-noise power available from the secondary power circuit 318, device performance may be improved.

According to an embodiment of the present invention, if the main power supply 312 is shut down, depleted or removed, such as, for example, when a battery is removed, data may be backed up to the memory 320 and the switch 316 may be opened via its control line. In this embodiment, the circuitry 314 would be without power but the memory 320 would be powered, possibly for an extended period of time, by the secondary power circuit 318.

The secondary power circuit 318 may be a variety of power storage devices. For example, the power storage device may be a battery, a capacitor or some other storage device. If the power storage device is a capacitor, the capacitor may be a double layer capacitor and may have a low ESR. In addition, the capacitor may have a high value for high energy storage, may have a significantly long expiration date and may support a large number of charge-discharge cycles. The switch 316 may be any of a variety of switches. For example, the switch 316 may be an analog switch, a bipolar transistor, a FET, a MOS transistor and the like.

Referring back to FIG. 1 and FIG. 2, the external device may communicate in a variety of ways. For example, the external device 16 may communicate telemetrically with the patient device 12 or may communicate with a patient or other user using its display 22. In addition, the external device 16 may communicate by generating an audible signal. For example, according to an embodiment of the present invention, if the external device 16 is configured to communicate with an patient device 12 configured as an insulin pump with a glucose sensor, and the internal device detects an alarm condition in a patient, the internal device may generate an alarm signal and communicate the alarm signal to the external device 16. The external device 16 may then generate an audible alarm to alert the patient that an alarm condition exists.

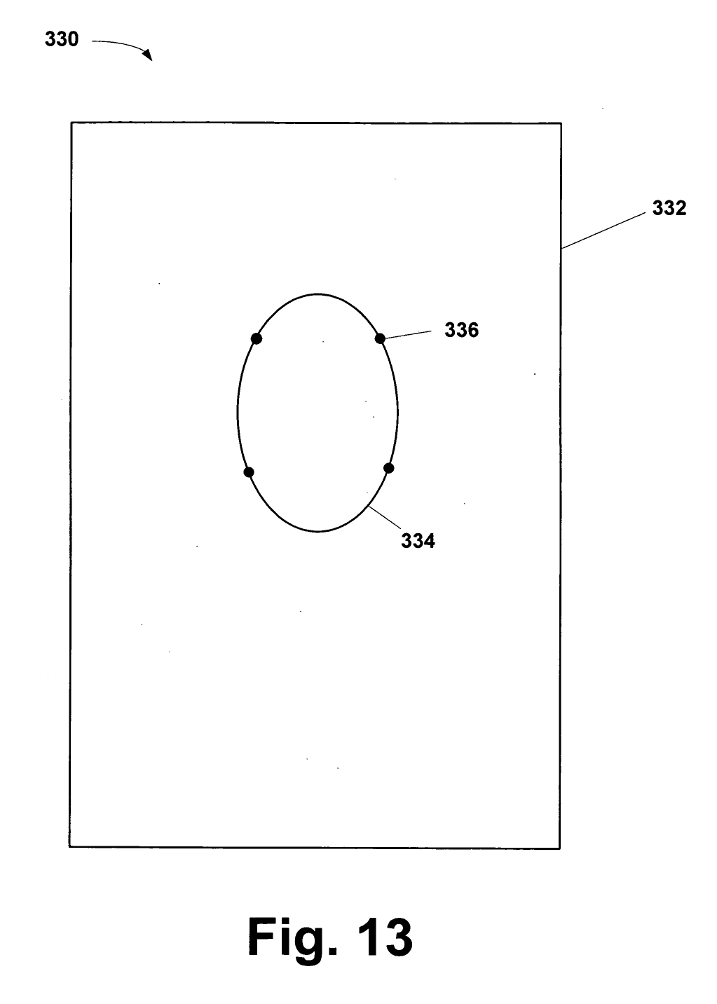

FIG. 13 shows a block diagram of a rear portion 332 of an external device 330 upon which is mounted a transducer 334 according to an embodiment of the present invention. The transducer 334 may be maintained in place on the rear portion 332 or any other portion of the external device 330 by pins 336. The transducer 334 may be connected to circuitry or other hardware that generates a signal that is received by the transducer. The transducer may be any of a variety of transducers, such as an acoustic or audible transducer, for example.

According to embodiments of the present invention, the pins 336 may be made from plastic, metal or other material that may be melted. The pins 336 may actually be an extrusion, protrusion, projection or the like that extends outwardly from the external device 330 toward the transducer 334. The pins 336 may also be a lip that encircles or partially encircles the transducer 334. The term “pins” here is used simply for convenience. In addition, the pins 336 may take a variety of shapes. According to embodiments of the present invention, the pins 336 may be rectangular, square, cylindrical or the like or may be configured in a random way so long as they are sufficient to support the transducer 334.

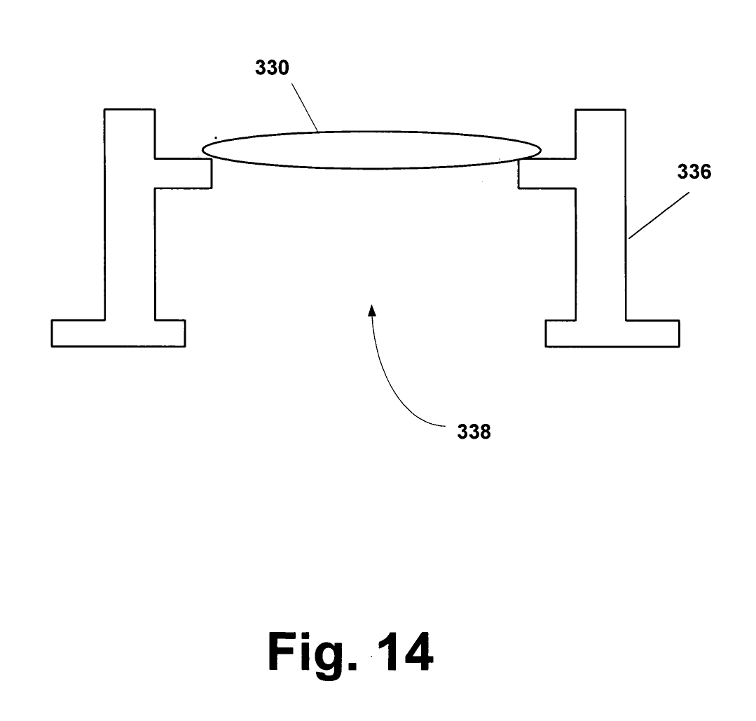

FIG. 14 shows a side view of the transducer 334 and pins 336. According to the embodiment of the invention shown in FIG. 14, the transducer is mounted on the pins prior to melting the pins. The pins 336 may be configured such that a chamber 338, such as an acoustic chamber, for example, forms under the transducer 334. The chamber 338 may enhance any sound generated by the transducer 334.

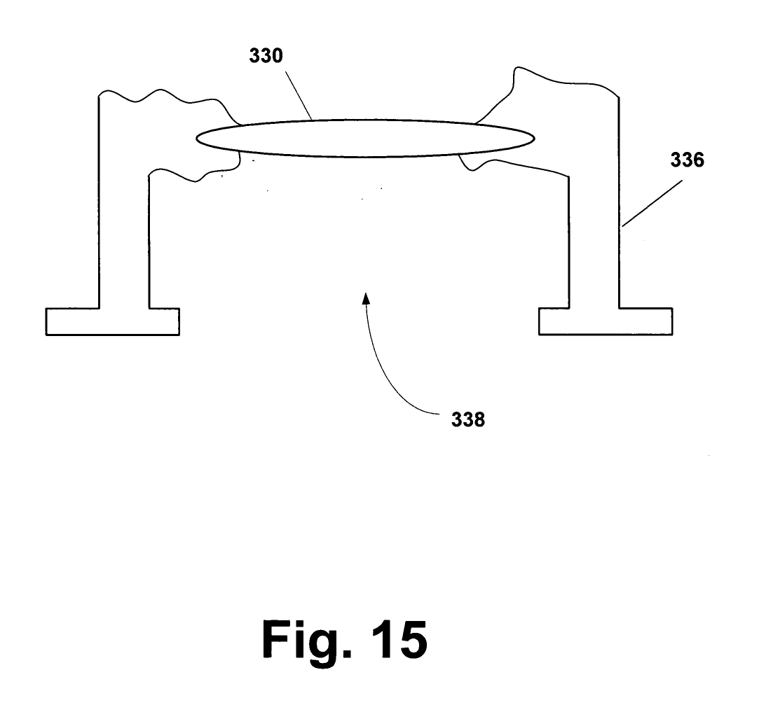

FIG. 15 shows a side view of the transducer 334 and melted pins 336. According to an embodiment of the present invention, the pins 336 may be plastic and may be melted so that they form around the edges of the transducer 334 or overlap a portion of the transducer 334. When the pins 336 cool and harden, the transducer is maintained in its position relative to the external device 330. The pins 336 may be melted by heat, ultrasonics, vibrations, friction and the like.

While particular embodiments of the present invention have been shown and described, it will be obvious to those skilled in the art that the invention is not limited to the particular embodiments shown and described and that changes and modifications may be made without departing from the spirit and scope of the appended claims.

1. A medical device comprising:
communication circuitry configured to process data transmitted to or received from an implantable device; and
a plurality of antennas connected to the communication circuitry,
wherein the plurality of antennas are disposed in the medical device such that they communicate in a plurality of directions.

2. The medical device of claim 1, wherein the plurality of antennas are disposed at specified angles in specified planes relative to each other.

3. The medical device of claim 1, wherein the plurality of antennas communicate with the implantable device via magnetic coupling.

4. The medical device of claim 1, wherein the plurality of antennas communicate with the implantable device using a wavelength in the kilometer range.

5. The medical device of claim 1, wherein the plurality of antennas communicate with the implantable device using a frequency in the kilohertz range.

6. The medical device of claim 5, wherein the frequency used by the plurality of antennas to communicate with the implantable device is about 131 kilohertz.

7. The medical device of claim 1, wherein the plurality of antennas includes a first antenna and a second antenna.

8. The medical device of claim 7, wherein the first antenna and the second antenna are disposed substantially perpendicularly to one another.

9. The medical device of claim 1, wherein the plurality of antennas includes a first antenna, a second antenna and a third antenna.

10. The medical device of claim 9, wherein the first antenna, the second antenna and the third antenna are disposed substantially perpendicularly to one another.

11. The medical device of claim 9, wherein the first antenna, the second antenna and the third antenna are disposed such that the medical device communicates in three dimensions.

12. The medical device of claim 1, wherein the plurality of antennas are configured as a plurality of inductors.

13. The medical device of claim 1, further comprising a plurality of amplifiers connected to the plurality of antennas for driving the antennas.

14. The medical device of claim 13, wherein the plurality of amplifiers include enabling and disabling circuitry.

15. The medical device of claim 14, wherein the enabling and disabling circuitry includes tri-state circuitry.

16. The medical device of claim 13, wherein the plurality of amplifiers are configured to drive only one antenna of the plurality of antennas at a time.

17. The medical device of claim 13, wherein the communication circuitry is configured to select at least one amplifier among the plurality of amplifiers to drive at least one antenna among the plurality of antennas.

18. The medical device of claim 13, wherein the plurality of amplifiers are linear amplifiers.

19. The medical device of claim 13, wherein the plurality of amplifiers are logic buffers.

20. The medical device of claim 12, further comprising a plurality of capacitors for tuning the plurality of inductors.

21. The medical device of claim 1, wherein the implantable device is disposed internally in a patient.

22. The medical device of claim 13, wherein the communication circuitry is configured to enable the plurality of amplifiers to consecutively select each antenna among the plurality of antennas until a strong signal is detected on at least one antenna of the plurality of antennas.

23. The medical device of claim 13, wherein the communication circuitry is configured to enable the plurality of amplifiers to consecutively select each antenna among the plurality of antennas for transmission of a signal until an acknowledgement is received indicating transmission has been successful.

24. The medical device of claim 23, wherein the communication circuitry is configured to enable the plurality of amplifiers to select the at least one antenna of the plurality of antennas as a first antenna for transmission or reception of a subsequent signal following detection of the strong signal.

25. The medical device of claim 9, the communication circuitry is configured to:
select the first antenna during a first time slot:
select a second antenna during a second time slot subsequent to the first time slot; and
select a third antenna during a third time slot subsequent to the first time slot and the second time slot.

26. A medical device system comprising:
an implantable unit for implanting into a patient;
an external unit for communicating with the implantable unit; and
a plurality of antennas disposed in the external unit such that the plurality of antennas communicate in a plurality of directions.

27. The medical device system of claim 26, wherein the plurality of antennas are disposed at specified angles in specified planes relative to each other.

28. The medical device system of claim 26, wherein the plurality of antennas communicate with the implantable unit via magnetic coupling.

29. The medical device system of claim 26, wherein the plurality of antennas communicate with the implantable unit using a wavelength in the kilometer range.

30. The medical device system of claim 26, wherein the plurality of antennas communicate with the implantable unit using a frequency in the kilohertz range.

31. The medical device system of claim 26, wherein the frequency used by the plurality of antennas to communicate with the implantable unit is about 131 kilohertz.

32. The medical device system of claim 26, wherein the plurality of antennas includes a first antenna and a second antenna.

33. The medical device system of claim 32, wherein the first antenna and the second antenna are disposed in the external unit substantially perpendicularly to one another.

34. The medical device system of claim 26, wherein the plurality of antennas includes a first antenna, a second antenna and a third antenna.

35. The medical device system of claim 34, wherein the first antenna, the second antenna and the third antenna are disposed in the external unit substantially perpendicularly to one another.

36. The medical device system of claim 34, wherein the first antenna, the second antenna and the third antenna are disposed in the external unit such that the external unit communicates with the implantable unit in three dimensions.

37. The medical device system of claim 26, wherein the plurality of antennas are configured as a plurality of inductors.

38. The medical device system of claim 26, further comprising a plurality of amplifiers connected to the plurality of antennas for driving the antennas.

39. The medical device system of claim 38, wherein the plurality of amplifiers include enabling and disabling circuitry.

40. The medical device system of claim 39, wherein the enabling and disabling circuitry includes tri-state circuitry.

41. The medical device system of claim 38, wherein the plurality of amplifiers are configured to drive only one antenna of the plurality of antennas at a time.

42. The medical device system of claim 38, further comprising circuitry disposed in the external unit configured to select at least one amplifier among the plurality of amplifiers to drive at least one antenna among the plurality of antennas.

43. The medical device system of claim 38, wherein the plurality of amplifiers are linear amplifiers.

44. The medical device system of claim 38, wherein the plurality of amplifiers are logic buffers.

45. The medical device system of claim 37, further comprising a plurality of capacitors for tuning the plurality of inductors.

46. The medical device system of claim 26, wherein the implantable unit is disposed internally in a patient.

47. The medical device system of claim 42, wherein the circuitry is configured to enable the plurality of amplifiers to consecutively select each antenna among the plurality of antennas until a strong signal is detected on the at least one antenna of the plurality of antennas.

48. The medical device system of claim 42, wherein the circuitry is configured to enable the plurality of amplifiers to consecutively select each antenna among the plurality of antennas for transmission of a signal until an acknowledgement is received indicating transmission has been successful.

49. The medical device system of claim 47, wherein the circuitry is configured to enable the plurality of amplifiers to select the at least one antenna of the plurality of antennas as a first antenna for transmission or reception of a subsequent signal following detection of the strong signal.

50. A method of communication in a medical device comprising:
providing communication circuitry in the medical device, the communication circuitry being configured to process data transmitted to or received from an implantable device;
disposing a plurality of antennas in the medical device;
connecting the plurality of antennas to the communication circuitry; and
communicating using the medical device,
wherein the plurality of antennas are disposed in the medical device such that they communicate in a plurality of directions.

51. The method of communication of claim 50, wherein communicating using the medical device includes communicating with an implantable device.

52. The method of communication of claim 51, wherein the implantable device is implanted in a patient.

53. The method of communication of claim 50, wherein communicating using the medical device includes communicating via magnetic coupling.

54. The method of communication of claim 50, wherein communicating using the medical device includes communicating using a frequency in the kilohertz range.

55. The method of communication of claim 50, wherein disposing a plurality of antennas in the medical device includes disposing two antennas.

56. The method of communication of claim 50, wherein disposing a plurality of antennas in the medical device includes disposing three antennas.

57. A medical device comprising:
communication circuitry means provided in the medical device, the communication circuitry being configured to process data transmitted to or received from an implantable device;
a plurality of antennas means disposed in the medical device and coupled to the communication circuitry; and
means for communicating using the medical device,
wherein the plurality of antennas are disposed in the medical device such that they communicate in a plurality of directions

58. A method of communication for a medical device comprising:
monitoring a first antenna in the medical device during a first time slot for a first transmitted signal;
monitoring a second antenna in the medical device during a second time slot for the first transmitted signal after monitoring for the first transmitted signal on the first antenna; and
generating an acknowledgement when a signal is detected on the first antenna or the second antenna.

59. The method of claim 27, further comprising monitoring a third antenna in the medical device during a third time slot for the first transmitted signal after monitoring for the first transmitted signal on the first antenna and the second antenna; and
generating an acknowledgement when a signal is detected on the third antenna.

60. The method of claim 59, further comprising continuing to monitor the first antenna, the second antenna and the third antenna during the first time slot, the second time slot and the third time slot, respectively, until the first transmitted signal is detected.

61. The method of claim 58, further comprising designating an antenna on which the first transmitted signal has been detected as a default antenna for subsequent monitoring.

62. The method of claim 58, further comprising receiving a second signal indicating that the acknowledgment was received.

63. The method of claim 59, wherein the first time slot, the second time slot and the third time slot occur at two-second intervals.

64. The method of claim 59, wherein the first antenna, the second antenna and the third antenna are disposed in the medical device substantially perpendicularly to one another.

65. A method of communication for a medical device comprising:
transmitting a first signal from a first unit in the medical device;
monitoring a first antenna in a second unit of the medical device during a first time slot for an acknowledgement that the first signal was received; and
monitoring a second antenna in the second unit of the medical device during a second time slot for an acknowledgement that the first signal was received.

66. The method of claim 65, further comprising monitoring a third antenna in the second unit of the medical device during a third time slot for an acknowledgement that the first signal was received.

67. The method of claim 65, further comprising receiving a signal indicating that the acknowledgment was received.

68. The method of claim 66, wherein the first time slot, the second time slot and the third time slot occur at two-second intervals.

69. The method of claim 66, wherein the first antenna, the second antenna and the third antenna are disposed in the medical device substantially perpendicularly to one another.

70. The method of claim 65, wherein the first unit is an implantable unit and the second unit is an external unit.

71. A method of communication for a medical device comprising:
monitoring a first antenna in a first unit of the medical device during a first time slot for a first signal;
monitoring a second antenna in the first unit of the medical device during a second time slot for the first signal after monitoring for the first signal on the first antenna; and
transmitting a request signal from the first unit of the medical device to a second unit of the medical device when the first signal has been detected;
generating an acknowledgement when the first signal is detected on the first antenna or the second antenna.

72. The method of claim 71, further comprising monitoring a third antenna in the first unit of the medical device during a third time slot for the first signal after monitoring for the first signal on the first antenna and the second antenna; and
generating an acknowledgement when the first signal is detected on the third antenna.

73. The method of claim 72, further comprising designating an antenna on which the first signal has been detected as a default antenna for subsequent monitoring.

74. The method of claim 72, wherein the first antenna, the second antenna and the third antenna are disposed in the medical device substantially perpendicular to one another.

75. A method for supplying power to a circuit comprising:
providing a first power supply for supplying primary power to the circuit;
providing a second power supply for supplying secondary power to the circuit;
supplying the circuit with primary power during a non-communication process performed by the circuit;
temporarily disabling the first power supply during a communication process performed by the circuit; and
supplying the circuit with secondary power during the communication process.

76. The method for supplying power of claim 75, wherein an amount of noise introduced into the circuit by the second power supply is less than an amount of noise introduced into the circuit by the first power supply.

77. The method for supplying power of claim 75, wherein the first power supply is a switching regulator.

78. The method for supplying power of claim 75, wherein the second power supply is a battery.

79. The method for supplying power of claim 75, wherein the second power supply is a capacitor.

80. The method for supplying power of claim 79, wherein the capacitor is a double layer capacitor.

81. The method for supplying power of claim 75, wherein the first power supply charges the second power supply.

82. The method for supplying power of claim 75, wherein supplying the circuit with secondary power during the communication process includes temporarily powering the circuit with secondary power when the circuit is receiving a transmission.

83. The method for supplying power of claim 75, wherein supplying the circuit with secondary power during the communication process includes temporarily powering the circuit with secondary power when the circuit is sending a transmission.

84. A system for supplying power to a circuit comprising:
a first power supply for supplying primary power to the circuit; and
a second power supply for supplying secondary power to the circuit,
wherein the first power supply supplies the circuit with primary power during a non-communication process performed by the circuit, and
wherein the second power supply supplies the circuit with secondary power during the communication process.

85. The system of claim 84, wherein the first power supply charges the second power supply.

86. The system of claim 84, wherein the first power supply is a switching regulator.

87. The system of claim 84, wherein the second power supply is a capacitor.

88. The system of claim 87, wherein the capacitor is a double layer capacitor.

89. The system of claim 50, wherein the second power supply is a battery.

90. The system of claim 84, wherein an amount of noise introduced into the circuit by the second power supply is less than an amount of noise introduced into the circuit by the first power supply.

91. The system of claim 84, wherein the communication process is a transmission.

92. The system of claim 84, wherein the communication process is a reception.

93. The system of claim 84, wherein the first power supply is a switching regulator and the second power supply is a capacitor.

94. A method of affixing a transducer to a device comprising:
providing at least one pin fixedly attached to the device;
disposing the transducer on the device adjacent the at least one pin; and
melting at least a portion of the at least one pin,
wherein the at least one pin is melted such that at least a portion of the at least one pin after melting overlaps a portion of the transducer to clasp a portion of the transducer.

95. The method of claim 94, wherein providing at least one pin includes providing a plastic pin.

96. The method of claim 94, wherein providing at least one pin includes providing a metal pin.

97. The method of claim 94, wherein melting the at least one pin includes melting with heat.

98. The method of claim 94, wherein melting the at least one pin includes melting with ultrasonics.

99. The method of claim 94, wherein melting the at least one pin includes melting with friction.

100. The method of claim 94, wherein melting the at least one pin includes melting with vibrations.

101. The method of claim 94, further comprising forming a chamber when the transducer is disposed on the device.

102. The method of claim 101, wherein the chamber is an acoustic chamber.

103. A system for affixing a transducer onto a device comprising:
means for providing at least one pin fixedly attached to the device;
means for disposing the transducer on the device adjacent the at least one pin; and
means for melting the at least one pin,
wherein the at least one pin is melted such that at least a portion of the at least one pin after melting overlaps a portion of the transducer to clasp a portion of the transducer.

104. The system of claim 103, wherein the at least one pin is plastic.

105. The system of claim 103, wherein the at least one pin is metal.

106. The system of claim 103, wherein the means for melting the at least one pin is a heat means.

107. The system of claim 103, wherein the means for melting the at least one pin is an ultrasonics means.

108. The system of claim 103, wherein the means for melting the at least one pin is a friction means.

109. The system of claim 103, wherein the means for melting the at least one pin is a vibration means.

110. The system of claim 103, wherein the transducer is disposed on the device such that it forms a chamber with the device.

111. The system of claim 110, wherein the chamber is an acoustic chamber.

112. A medical device comprising:
communication circuitry configured to process data transmitted to or received from an implantable device;
a plurality of antennas connected to the communication circuitry;
at least one pin fixedly attached to the medical device; and
a transducer disposed on the medical device adjacent the at least one pin,
wherein the plurality of antennas are disposed in the medical device such that they communicate in a plurality of directions, and
wherein the at least one pin is melted such that at least a portion of the at least one pin after melting overlaps a portion of the transducer to clasp a portion of the transducer.

113. The medical device of claim 112, wherein the plurality of antennas are disposed at specified angles in specified planes relative to each other.

114. The medical device of claim 112, wherein the plurality of antennas communicate with the implantable device via magnetic coupling.

115. The medical device of claim 112, wherein the plurality of antennas communicate with the implantable device using a wavelength in the kilometer range.

116. The medical device of claim 112, wherein the plurality of antennas communicate with the implantable device using a frequency in the kilohertz range.

117. The medical device of claim 116, wherein the frequency used by the plurality of antennas to communicate with the implantable device is about 131 kilohertz.

118. The medical device of claim 112, wherein the plurality of antennas includes a first antenna, a second antenna and a third antenna.

119. The medical device of claim 118, wherein the first antenna, the second antenna and the third antenna are disposed substantially perpendicularly to one another.

120. The medical device of claim 118, wherein the first antenna, the second antenna and the third antenna are disposed such that the medical device communicates in three dimensions.

121. The medical device of claim 112, wherein the plurality of antennas are configured as a plurality of inductors.

122. The medical device of claim 112, wherein the implantable device is disposed internally in a patient.
